SEQUENCE LISTING (43 sequences)

SEQ ID NO: 1 - DNA, 1215 bp, Brassica napus
atggcgaatgcatctggatttttcacccatcccgtacccagaaatttgggatctaaaattcacgtgcctgtaggactatcaggaagcggtttttgtgtgagtctctttatatctaaaagagttctctgttcttctgtggaggttgacaaggatgctagcgcctctccttctcgtagcgagtatcaacgtcctttagtttctcgcggttgcaagctaattggatgtggatcagcagttccaagtcttctgatttctaatgatgatctcgctaaaatagttgatactaatgatgaatggattgctactcgtactggtattcgcacccgtcgagttgtatcagccaaagatagcttggttggcttagcagtagaagcagcaaccaaagctcttgaaatggctgaggttgttcctgaagatattgacttagtcttgatgtgtacttccactcctgatgatctgtttggtgctgctccacagattcaaaaggcacttggttgcacaaagaacccattggcttatgatatcacagctgcttgtagtggatttgttttgggtctagtttcagctgattgtcatataagaggaggtggttttaagaatgttttagtgattggagctgattctttatctcggtttgttgattggactgatagaggatcttgcatcctctttggggacgccgctggtgctgttgttgttcaggcatgcgatattgaggatgatgggttatttagtttcgatgtacacagcgatggagatggtcgtaggcatttgaatgcttctattaaagactcccaaaccaatggtgagttgagctccaacggatctgtgttgggagacttccaaccgagacaagcttcatattcttgcattcagatgaatggaaaagaggtctttcgctttgctgtcaaatgtgttcctcaatctattgaatctgctttacaaaaagccggtcttcctgcttctgccatcgactggctcctcctccaccaggcgaaccagagaataatagactctgtggctacaagtctgcatttcccaccagagcgagtcatatcgaatttggctaattacggtaacacgagcgctgcttcgattccgctggctcttgatgaggcagtgagaagcggaaaagttaaaccaggacataccatagcgacatccggttttggagccggtttaacgtggggatcagcaattgtcaagtggggttga

SEQ ID NO: 2 - protein, 404 aa, Brassica napus
MANASGFFTHPVPRNLGSKIHVPVGLSGSGFCVSLFISKRVLCSSVEVDKDASASPSRSEYQRPLVSRGCKLIGCGSAVPSLLISNDDLAKIVDTNDEWIATRTGIRTRRVVSAKDSLVGLAVEAATKALEMAEVVPEDIDLVLMCTSTPDDLFGAAPQIQKALGCTKNPLAYDITAACSGFVLGLVSADCHIRGGGFKNVLVIGADSLSRFVDWTDRGSCILFGDAAGAVVVQACDIEDDGLFSFDVHSDGDGRRHLNASIKDSQTNGELSSNGSVLGDFQPRQASYSCIQMNGKEVFRFAVKCVPQSIESALQKAGLPASAIDWLLLHQANQRIIDSVATSLHFPPERVISNLANYGNTSAASIPLALDEAVRSGKVKPGHTIATSGFGAGLTWGSAIVKWG

SEQ ID NO: 3 - DNA, 1209 bp, Cuphea lanceolata
atggcgaatgctttggggtttgtgggtcattcagttccaaccatgggaagggcagctcagtttcagcagatgggatctgggttttgttctgcagacttcatttccaagagggtgttttgttgcagtgtcgttcaaggcgctggcaagccggcctcgggtgattctcgtaccgaatatcgaacgccgaggtttgtgagtagaggatgcaaattggttggatctggttcggctataccagctcttcaagtctcaaatgacgatcttgcaaagattgttgataccaatgatgaatggatttctgtccgaacaggaatccgcaatcgacgggttctaactggtaaagatagtcttacaaatttagcaacagaggcagcgaggaaagctctagagatggcgcaggttgatgcaaatgatgtggatatggtattgatgtgcacctcaacccccgaggatctctttggtagtgctcctcagattcagaaggcacttggttgcaagaagaatcctttggcttatgatatcaccgccgcgtgcagtggatttgtgttgggtcttgtatcagctgcttgccatattagaggtggtggatttaacaatattctagtgattggtgctgattccctctctcggtatgttgactggaccgatcgaggaacctgtattctctttggagatgcagccggtgctgtgcttgttcagtcctgtgatgctgaggaagatgggctctttgcttttgatttgcacagcgatggagacgggcagaggcacctaaaagccgcaattacagaaaacgaaattgatcatgccgtgggaactaatggatccgtgtcagattttccaccaggacgttcttcatattcttgcatccaaatgaatggtaaagaggtcttccgctttgcttgccgttccgtgcctcagtcaattgaatcagctcttggaaaggccggtcttaatggatccaacatcgactggctactgcttcatcaggcgaatcagaggatcatcgatgcagttgcgacacgtctagaggttcctcgggagcgagtgatctcaaacttggcgaattatgggaacacaagcgctgcatctatccccttggcacttgacgaagctgttcggggtgggaaggtgaaggccggccacctgatagctacagcaggattcggcgcgggactcacttggggttctgccattgtcagatggggatga

SEQ ID NO: 4 - protein, 402 aa, Cuphea lanceolata
MANALGFVGHSVPTMGRAAQFQQMGSGFCSADFISKRVFCCSVVQGAGKPASGDSRTEYRTPRFVSRGCKLVGSGSAIPALQVSNDDLAKIVDTNDEWISVRTGIRNRRVLTGKDSLTNLATEAARKALEMAQVDANDVDMVLMCTSTPEDLFGSAPQIQKALGCKKNPLAYDITAACSGFVLGLVSAACHIRGGGFNNILVIGADSLSRYVDWTDRGTCILFGDAAGAVLVQSCDAEEDGLFAFDLHSDGDGQRHLKAAITENEIDHAVGTNGSVSDFPPGRSSYSCIQMNGKEVFRFACRSVPQSIESALGKAGLNGSNIDWLLLHQANQRIIDAVATRLEVPRERVISNLANYGNTSAASIPLALDEAVRGGKVKAGHLIATAGFGAGLTWGSAIVRWG

SEQ ID NO: 5 - DNA, 1209 bp, Cuphea lanceolata
atggcgaatgctttggggtttgtgggtcattcagttccaaccatgggaagggcagctcagtttcagcagatgggatctgggttttgttctgcagacttcatttccaagagggtgttttgttgcagtgtcgttcaaggcgctggcaagccggcctcgggtgattctcgtaccgaatatcgaacgccgaggtttgtgagtagaggatgcaaattggttggatctggttcggctataccagctcttcaagtctcaaatgacgatcttgcaaagattgttgataccaatgatgaatggatttctgtccgaacaggaatccgcaatcgacgggttctaactggtaaagatagtcttacaaatttagcaacagaggcagcgaggaaagctctagagatggcgcaggttgatgcaaatgatgtggatatggtattgatgtgcacctcaacccccgaggatctctttggtagtgctcctcagattcagaaggcacttggttgcaagaagaatcctttggcttatgatatcaccgccgcgtgcagtggatttgtgttgggtcttgtatcagctgcttgccatattagaggtggtggatttaacaatattctagtgattggtgctgattccctctctcggtatgttgactggaccgatcgaggaacctgtattctctttggagatgcagccggtgctgtgcttgttcagtcctgtgatgctgaggaagatgggctctttgcttttgatttgcacagcgatggagacgggcagaggcacctaaaagccgcaattacagaaaacgaaattgatcatgccgtgggaactaatggatccgtgtcagattttccaccaggacgttcttcatattcttgcatccaaatgaatggtaaagaggtcttccgctttgcttgccgttccgtgcctcagtcaattgaatcagctcttggaaaggccggtcttaatggatccaacatcgactggctactgcttcatcaggcgaatcagaggatcatcgatgcagttgcgacacgtctagaggttcctcgggagcgagtgatctcaaacttggcgaattatggggacacaagcgctgcatctatccccttggcacttgacgaagctgttcggggtgggaaggtgaaggccggccacctgatagctacagcaggattcggcgcgggactcacttggggttctgccattgtcagatggggatga

SEQ ID NO: 6 - protein, 402 aa, Cuphea lanceolata
MANALGFVGHSVPTMGRAAQFQQMGSGFCSADFISKRVFCCSVVQGAGKPASGDSRTEYRTPRFVSRGCKLVGSGSAIPALQVSNDDLAKIVDTNDEWISVRTGIRNRRVLTGKDSLTNLATEAARKALEMAQVDANDVDMVLMCTSTPEDLFGSAPQIQKALGCKKNPLAYDITAACSGFVLGLVSAACHIRGGGFNNILVIGADSLSRYVDWTDRGTCILFGDAAGAVLVQSCDAEEDGLFAFDLHSDGDGQRHLKAAITENEIDHAVGTNGSVSDFPPGRSSYSCIQMNGKEVFRFACRSVPQSIESALGKAGLNGSNIDWLLLHQANQRIIDAVATRLEVPRERVISNLANYGDTSAASIPLALDEAVRGGKVKAGHLIATAGFGAGLTWGSAIVRWG

SEQ ID NO: 7 - DNA, 20 bp, Artificial Sequence
  A primer
tggaaaggccggccttaatg

SEQ ID NO: 8 - DNA, 21 bp, Artificial Sequence
  A primer
ctcgagttatccccacctgat

SEQ ID NO: 9 - DNA, 20 bp, Artificial Sequence
  A primer
aactacggggacactagtgc

SEQ ID NO: 10 - DNA, 20 bp, Artificial Sequence
  A primer
gcactagtgtccccgtagtt

SEQ ID NO: 11 - DNA, 21 bp, Artificial Sequence
  A primer
aacactagttcggcatccatt

SEQ ID NO: 12 - DNA, 21 bp, Artificial Sequence
  A primer
aatggatgccgaactagtgtt

SEQ ID NO: 13 - DNA, 20 bp, Artificial Sequence
  A primer
cactagtgcgccatccattc

SEQ ID NO: 14 - DNA, 20 bp, Artificial Sequence
  A primer
gaatggatggcgcactagtg

SEQ ID NO: 15 - DNA, 19 bp, Artificial Sequence
  A primer
gcaaactacgcggcatcca

SEQ ID NO: 16 - DNA, 19 bp, Artificial Sequence
  A primer
tggatgccgcgtagttggc

SEQ ID NO: 17 - DNA, 28 bp, Artificial Sequence
  A primer
aactggaagaattcgcggccgcaggaat

SEQ ID NO: 18 - DNA, 20 bp, Artificial Sequence
  A primer
atggcnaaygcntynggstt

SEQ ID NO: 19 - DNA, 20 bp, Artificial Sequence
  A primer
atyctctgrttngcytgrtg

SEQ ID NO: 20 - DNA, 21 bp, Artificial Sequence
  A primer
gaygtngayatggtnytnatg

SEQ ID NO: 21 - DNA, 17 bp, Artificial Sequence
  A primer
ayaatngcnccccangt

SEQ ID NO: 22 - DNA, 27 bp, Artificial Sequence
  A primer
ttcctgcggccgcgaattcttccagtt

SEQ ID NO: 23 - DNA, 21 bp, Artificial Sequence
  A primer
catagcgatggagatgggcaa

SEQ ID NO: 24 - DNA, 21 bp, Artificial Sequence
  A primer
cattcagatggcgatggtcag

SEQ ID NO: 25 - DNA, 21 bp, Artificial Sequence
  A primer
catatgagaggatgcaaattg

SEQ ID NO: 26 - DNA, 21 bp, Artificial Sequence
  A primer
ctcgagtcatccccatctgac

SEQ ID NO: 27 - DNA, 21 bp, Artificial Sequence
  A primer
catatgcgcggttgcaagcta

SEQ ID NO: 28 - DNA, 21 bp, Artificial Sequence
  A primer
ctcgagtcaaccccacttgac

SEQ ID NO: 29 - DNA, 20 bp, Artificial Sequence
  A primer
atggcgaatgctttggggtt

SEQ ID NO: 30 - DNA, 20 bp, Artificial Sequence
  A primer
tcatccccatctgacaatgg

SEQ ID NO: 31 - DNA, 21 bp, Artificial Sequence
  A primer
gtgagtagaggatgcaaattg

SEQ ID NO: 32 - DNA, 21 bp, Artificial Sequence
  A primer
tcctctactcacaaacctcgg

SEQ ID NO: 33 - DNA, 33 bp, Artificial Sequence
  A primer
cttggcgaattatggggacacaagcgctgcatc

SEQ ID NO: 34 - DNA, 33 bp, Artificial Sequence
  A primer
gatgcagcgcttgtgtccccataattcgccaag

SEQ ID NO: 35 - protein, 406 aa, Brassica napus
MANSYGFFTPSVPRSLGNKAQVPVGLSGSGFCSSLFISKRVFCSSVSESEKDAPAGNSRSESRVSRLVSRGCKLVGCGSAVPSLQISNDDLSKFVETSDEWIATRTGIRTRRVLSGKDSLTNLAAEAARNALEMAQVNADDIDLILMCTSTPEDLFGSAPQIQRALGCKKNPLSYDITAACSGFVLGLVSAACHVRGGGFKNVLVIGADSLSRFVDWTDRGTCILFGDAAGAVLVQACDIEEDGLFSFDVHSDGDGRRHLNAAVKESRVDAALGSNGSVFRDFPPRQSSYSCIQMNGKEVFRFAVRCVPQSIEAALQKAGLPSSNIDWLLLHQANQRIIDAVSTRLEVPSERVISNLANYGNTSAASIPLALDEAVRSGKVKPGNTIATSGFGAGLTWGSAIIRWG

SEQ ID NO: 36 - protein, 402 aa, Cuphea lanceolata
MANALGFVGHSVPTMGRAAQFQQMGSGFCSADFISKRVFCCSVVQGAGKPASGDSRTEYRTPRFVSRGCKLVGSGSAIPALQVSNDDLAKIVDTNDEWISVRTGIRNRRVLTGKDSLTNLATEAARKALEMAQVDANDVDMVLMCTSTPEDLFGSAPQIQKALGCKKNPLAYDITAACSGFVLGLVSAACHIRGGGFNNILVIGADSLSRYVDWTDRGTCILFGDAAGAVLVQSCDAEEDGLFAFDLHSDGDGQRHLKAAITENEIDHAVGTNGSVRDFPPGRSSYSCIQMNGKEVFRFACRSVPQSIESALGKAGLNGSNIDWLLLHQANQRIIDAVATRLEVPRERVISNLANYGNTSAASIPLALDEAVRGGKVKAGHLIATAGFGAGLTWGSAIVRWG

SEQ ID NO: 37 - protein, 402 aa, Cuphea wrightii
MANAYGFVGHSVPTMKRAAQFQQMGSGFCSADSISKRVFCCSVVQGADKPASGDSRTEYRTPRLVSRGCKLVGSGSAMPALQVSNDDLSKIVDTNDEWISVRTGIRNRRVLTGKESLTNLATVAARKALEMAQVDANDVDMVLMCTSTPEDLFGSAPQIQKALGCKKNPLAYDITAACSGFVLGLVSAACHIRGGGFNNILVIGADSLSRYVDWTDRGTCILFGDAAGAVLVQSCDAEEDGLFAFDLHSDGDGQRHLKAAITENGIDHAVGSNGSVSDFPPRSSSYSCIQMNGKEVFRFACRCVPQSIESALGKAGLNGSNIDWLLLHQANQRIIDAVATRLEVPQERVISNLANYGNTSAASIPLALDEAVRGGKVKAGHLIATAGFGAGLTWGSAIVRWG

SEQ ID NO: 38 - protein, 400 aa, Cuphea wrightii
MANASGFLGSSVPALRRATQPQHSISSSRGSSSDFVFKRVFCCSAVQGSDRQSLGDSRSPRLVSRGCKLIGSGSAIPSLQISNDDLAKIVDTNDEWISVRTGIRNRRVLTGKDSLTNLASEAARKALEMAQIDADDVDMVLMCTSTPEDLFGSAPQISKALGCKKNPLSYDITAACSGFVLGLVSAACHIRGGGFNNVLVIGADSLSRYVDWTDRGTCILFGDAAGAVVVQSCDAEEDGLFAFDLHSDGDGQRHLKAAIKEDEVDKALGSNGSIRDFPPRRSSYSCIQMNGKEVFRFACRCVPQSIESALGKAGLNGSNIDWLLLHQANQRIIDAVATRLEVPQERIISNLANYGNTSAASIPLALDEAVRSGNVKPGHVIATAGFGAGLTWGSAIIRWG

SEQ ID NO: 39 - protein, 405 aa, Spinacia oleracea
MATSYGFFSPSVPSSLNNKISPSLGINGSGFCSHLGISKRVFCSSIEASEKHAAAGVSSSESRVSRLVNRGCKLVGCGSAVPKLQISNDDLSKFVETSDEWIATRTGIRQRHVLSGKDSLVDLAAEAARNALQMANVNPDDIDLILMCTSTPEDLFGSAPQVQRALGCSRTPLSYDITAACSGFMLGLVSAACHVRGGGFKNVLVIGADALSRFVDWTDRGTCILFGDAAGAVVVQACDSEEDGMFAFDLHSDGGGGRHLNASLLNDETDAAIGNNGAVTGFPPKRPSYSCINMNGKEVFRFAVRCVPQSIEAALQKAGLTSSNIDWLLLHQANQRIIDAVATRLEVPSERVLSNLANYGNTSAASIPLALDEAVRSGKVKPGNIIATSGFGAGLTWGSSIIRWG

SEQ ID NO: 40 - protein, 404 aa, Arabidopsis thaliana
MANASGFFTHPSIPNLRSRIHVPVRVSGSGFCVSNRFSKRVLCSSVSSVDKDASSSPSQYQRPRLVPSGCKLIGCGSAVPSLLISNDDLAKIVDTNDEWIATRTGIRTRRVVSAKDSLVGLAVEAATKALEMAEVVPEDIDLVLMCTSTPDDLFGAAPQIQKALGCTKNPLAYDITAACSGFVLGLVSADCHIRGGGFKNVLVIGADSLSRFVDWTDRGTCILFGDAAGAVVVQACDIEDDGLFSFDVHSDGDGRRHLNASVKESRNEGESSSNGSVFGDFPPKQSSYSCIQMNGKEVFRFAVKCVPQSIESALQKAGLPASAIDWLLLHQANQRIIDSVATSLHFPPERVISNLANYGNTSAASIPLALDEAVRSGKVKPGHTIATSGFGAGLTWGSAIVRWR

SEQ ID NO: 41 - protein, 401 aa, Allium sativum
MAAASIGFTTPSANPRIRARNFGNFGALGFLCFKERSFKRNWVGCCSVSESSSSLSYSTNRTKRLVGMGSKLIGSGSAVPKLQIFNDDMAKIVETSDEWISVRTGIRNRRVLTGNENLNGLAVEAAKGALRMAEVEAENVDLVIFWSSTPDDLFGGASRIQGDLGCKSALAFDITAACSGFVVGLITATRFIKGGGYKNVLVIGADALSRFVDWTDRGTCILFGDAAGAVLVQACSEDEDGLLGFDLNSDGSGQRHLNAFVSDAEHEAISNTNGAPLFPPKRSTYSCIKMNGNEVFRFGWRCVPQTIQASLDDAGLSSSNIDWLLLHQANQRIIDAVSTRLEIPSEKVISNLANYGNTSAASIPLALDEAVRNGKVKAGDTIATAGFGAGLTWGSAIVKWG

SEQ ID NO: 42 - protein, 326 aa, Porphyra umbilicalis
MGVHILSTGSSVPNFSVENQQFEDIIETSDHWISTRTGIKKSILPLLLPSLTKLAAEAANDALSKASINAEDIDLIILATSTPDDLFGSASQLQAEIGATSSTAFDITAACSGFIIALVTASQFIQAGSYNKVLVVGADTMSRWIDWSDRSSCILFGDGAGAVLIGESSINSILGFKLCTDGRLNSHLQLMNSPSDSQQFGLTTVPKGRYDSIRMNGNEVYKFAVFQVPIVIKNCLNDVNISIDEVDWFLLHQANIRILEAIATRLSIPLSKMITNLENYGNTSAASIPLALDEAIREKKIQPGQVVVLAGFGAGLTWGAIVLKWQ

SEQ ID NO: 43 - protein, 317 aa, Escherichia coli
MYTKIIGTGSYLPEQVRTNADLEKMVDTSDEWIVTRTGIRERHIAAPNETVSTMGFEAATRAIEMAGIEKDQIGLIVVATTSATHAFPSAACQIQSMLGIKGCPAFDVAAACAGFTYALSVADQYVKSGAVKYALVVGSDVLARTCDPTDRGTIIIFGDGAGAAVLAASEEPGIISTHLHADGSYGELLTLPNADRVNPENSIHLTMAGNEVFKVAVTELAHIVDKTLAANNLDRSQLDWLVPHQANLRIISATAKKLGMSMDNVVVTLDRHGNTSAASVPCALDEAVRDGRIKPGQLVLLEAFGGGFTWGSALVRF

(19) United States
(12) Patent Application Publication
(10) Pub. No.: US 2003/0145350 A1
(43) Pub. Date: Jul. 31, 2003

(54) Method for increasing the content of fatty acids in plants and micro-organisms

(76) Inventors: Friedrich Spener, Muenster (DE); Amine Abbadi, Hamm (DE); Monika Brummel, Muenster (DE)

Correspondence Address: SCHWEGMAN, LUNDBERG, WOESSNER & KLUTH, P.A., P.O. BOX 2938, MINNEAPOLIS, MN, 55402, US

(21) Appl. No.: 10/194,919

(22) Filed: Jul. 12, 2002

Related U.S. Application Data
(63) Continuation of application No. PCT/EP01/00289, filed on Jan. 11, 2001.

(30) Foreign Application Priority Data
Dec. 1, 2000 (DE) 100 00 978.6

(51) Int. Cl. A01H 5/00; C12Q 1/68; C07H 21/04; C12N 9/10; C12P 21/02; C12N 5/04

(52) U.S. Cl. 800/281; 435/6; 435/69.1; 435/320.1; 435/419; 435/193; 536/23.2

(57) ABSTRACT

The invention relates to DNA sequences which code for a protein having the enzymatic activity of a β-ketoacyl ACP synthase (KAS) of the enzyme complex of the fatty acid synthase (FAS). The invention also relates to transgenic plants and micro-organisms which contain nucleic acid sequences which code for proteins having the activity of a β-ketoacyl ACP ((acyl carrier protein)) synthase (KAS) of the enzyme complex of the fatty acid synthase (FAS). The invention further relates to a method for influencing the fatty acid pattern and/or for increasing the fatty acid content, especially the content of short and middle chain fatty acids, in plants, especially in seed tissues that synthesize and/or store triacylglycerols, as well as in micro-organisms, especially bacteria and algae. The inventive method comprises the expression of proteins having the activity of a KAS of the enzyme complex or the fatty acid synthase in transgenic plants or micro-organisms.

The invention relates to DNA sequences which code for a protein having the enzymatic activity of a β-ketoacyl ACP synthase (KAS) of the enzyme complex of the fatty acid synthase (FAS). The invention also relates to transgenic plants and micro-organisms which contain nucleic acid sequences that code for proteins having the activity of a β-ketoacyl ACP (acyl carrier protein) synthase of the enzyme complex of the fatty acid synthase. The invention further relates to a method of influencing the fatty acid pattern and/or increasing the fatty acid content, especially the content of short and middle chain fatty acids, in plants, especially in seed tissues and other tissues that synthesise and/or store triacylglycerols, as well as in micro-organisms, especially bacteria and algae. The inventive method comprises the expression of proteins having the activity of a KAS of the enzyme complex of the fatty acid synthase in transgenic plants or micro-organisms.

Due to compartmentation, the biosynthesis of fatty acids and triacylglycerols are considered to be distinct biosynthetic pathways, but with respect to the final product they may be regarded as a single biosynthetic pathway. De novo biosynthesis of fatty acids takes place within the plastids, and is catalysed essentially by three enzymes or enzyme systems, namely the acetyl CoA carboxylase, the fatty acid synthase and the acyl ACP thioesterases. The end products of said reaction sequence in most organisms are palmitate, stearate, and after a desaturation, oleate.

The fatty acid synthase consists of an enzyme complex of dissociable single enzymes comprising malonyl-CoA:ACP transferase; β-ketoacyl ACP synthases consisting of chain length-specific β-acyl ACP:malonyl ACP condensing enzymes (KAS I, II, IV) and the acetyl-CoA:malonyl ACP condensing enzyme (KAS III); β-ketoacyl ACP reductase; β-hydroxyacyl ACP dehydratase and enoyl ACP reductase.

The reaction of the fatty acid synthesis in seeds of oil seed plants starts with the reaction of acetyl CoA and malonyl ACP catalysed by KAS III, formation of the latter being catalysed by the malonyl-CoA:ACP transferase. In the subsequent steps of fatty acid synthesis the keto group of the formed β-ketobutyryl ACP is reduced to a methylene group, being first reduced to the D-β-hydroxybutyryl ACP and then crotonyl ACP is formed from D-β-hydroxybutyryl ACP by dehydration. By reducing crotonyl ACP to butyryl ACP in the final step of the cycle the first elongation cycle is completed. During the second cycle of the fatty acid synthesis butyryl ACP condenses with malonyl ACP to form C6-β-ketoacyl ACP. Subsequent reduction, dehydration and a second reduction convert the intermediate product C6-β-ketoacyl ACP into C6-acyl ACP, which is provided for a third elongation cycle. These elongation cycles continue to the formation of palmitoyl and stearoyl ACP. These products are hydrolysed to form palmitate, stearate and ACP, but wherein stearoyl ACP is mainly desaturated to form olcoyl ACP and is then also hydrolysed.

In synthesising short and middle chain fatty acids hydrolysis takes place by aid of acyl ACP thioesterases, which specifically act on short and middle chain acyl derivatives.

Biosynthesis of triacylglycerol from glycerol-3-phosphate and fatty acids, which have been activated to the acyl CoA substrates beforehand, takes place in the endoplasmatic reticulum in the so-called “Kennedy-pathway” after external transport of the fatty acids into the cytoplasm.

The term fatty acid is to be understood as saturated or unsaturated, short, middle or long chain, straight-chain or branched-chain, even-numbered or uneven-numbered fatty acids. Short chain fatty acids are generally meant to be fatty acids having up to 6 carbon atoms, such as, for example, butyric acid, valeric acid and caprylic acid. The term middle chain fatty acids includes C₈ to C₁₄ fatty acids, i.e. for example, caproic acid, lauric acid and myristic acid. Finally, long chain fatty acids comprise those which have at least 16 carbon atoms, such as palmitic acid, stearic acid, oleic acid, linoic acid and linoleic acid. However, often also C₄-C₈ fatty acids are denoted as short chain, and C₆-C₁₀ fatty acids as middle chain. Therefore, there are no strict definitions, but rather a classification with fluent transitions.

Fatty acids occurring in all plant and animal lipids and especially in plant oils and fish oils as well as in micro-organisms are widely applicable. For example, deficiency in essential fatty acids, i.e. fatty acids, which can not be synthesised in the organism and therefore are to be ingested by way of food, may lead to skin irritations und growth disturbances. This is the reason why fatty acids are applied in eczema, psoriasis, bums and the like, and why they are used in cosmetics. Furthermore, fatty acids and oils are used in washing and cleansing agents, as detergents, as colorant additives, lubricants and slip agents, processing agents, emulsifiers, hydraulic oils and as flotation oils in pharmaceutical and cosmetic products. Natural lipids and oils of animal origin (e.g. tallow) und plant origin (e.g. coconut oil, palm kernel oil or rape-seed oil) are used as renewable raw materials in the chemical-technical field. The applications for plant oils have considerably expanded during the last 20 years. Increasing ecological awareness have led to the development of environmentally compatible lubricants and hydraulic oils. Further applications for fatty acids and lipids are foodstuffs and nutritional supplements, e.g. in parenteral nutrition, in baking agents, in diets for babies, elderly people and athletes, in chocolate pastes, cocoa powder and as baking fats, for the manufacture of soaps, ointments, candles, painter's colours und textile paints, varnishes, fuels and illuminants.

A particular object of plant breeding is to increase the content of fatty acids in seed oils. Thus, as regards industrial rape and alternative production areas for agriculture, a breeding object is the production of rape-seed oil having middle chain fatty acids, since those are particularly desired in the manufacture of surfactants. Beside the idea of using plant oils as industrial raw materials, there is the possibility of using them as biological fuels.

Therefore, there is generally a need for providing fatty acids, which are industrially usable and/or are food-technologically useful as, for example, basic agents for softeners, lubricants, pesticides, surfactants, cosmetics and the like. One possibility for providing fatty acids is the extraction of the fatty acids from plants or micro-organisms having particularly high contents of the desired fatty acids. Increasing the content of e.g. middle chain fatty acids in plants by the conventional route, i.e. by means of breeding plants which synthesise these fatty acids to an increased degree, has been hitherto only partially successful. Hence, there is a particular interest in modern biotechnological attempts in plant breeding. Thus, for example, nucleic acids coding for proteins having the activity of the β-ketoacyl ACP synthases I, II, and IV, are known from the German patent application No. 199 26 456.2. Plants containing these nucleic acids have on the whole an increased content of fatty acids.

It is therefore an object of the present invention to provide transgenic plants and micro-organisms which synthesise fatty acids, which are synthesised only to a lesser extent or not at all in their wild-types. Especially, it is an object of the invention to provide plants and micro-organisms which show an increased content of short and middle chain fatty acids as compared to wild-type plants and/or micro-organisms.

Thus, it is also an essential object of the present invention to provide DNA sequences that code for proteins which influence the fatty acid pattern and/or the fatty acid content in plants and/or micro-organisms due to their enzymatic activity.

A further object is to provide methods for increasing the content of fatty acids, especially the content of short and middle chain fatty acids in plants, here especially in seed tissues and other tissues that synthesise and/or store triacylglycerols, as well as in micro-organisms, especially in bacteria and algae.

The features of the independent claims serve to solve these problems.

Advantageous embodiments are defined in the respective subclaims.

We have now succeeded in classifying an exact substrate specificity for the enzyme β-ketoacyl ACP synthase III (KAS III) which is involved in the fatty acid biosynthesis. KAS III catalyses the condensation of acetyl CoA with malonyl ACP to form β-ketobutyryl ACP, which is reduced to butyryl ACP by the following effect of an enzyme cascade. The product of this first elongation cycle represents the substrate for the condensation with malonyl ACP in the subsequent cycles, which are catalysed by several acyl ACP-specific condensing enzymes. In conventional plants this leads to an enrichment of primarily C₁₆- and C₁₈-acyl ACPs, which are hereinafter hydrolysed by an acyl ACP thioesterase.

In particular, the present invention provides, for the first time, a DNA sequence which encodes a protein having the enzymatic activity of a KAS III from Brassica napus.

In a preferred embodiment the DNA sequence encoding a protein, which has the enzymatic activity of a KAS III from Brassica napus, is selected from the group consisting of

a) DNA sequences comprising a nucleotide sequence, which code for the amino acid sequence identified in SEQ ID NO: 2 or fragments thereof,

b) DNA sequences comprising the nucleotide sequence identified in SEQ ID NO: 1, or parts thereof,

c) DNA sequences comprising a nucleotide sequence, which hybridises to a complementary strand of the nucleotide sequence of a) or b), or parts of said nucleotide sequence,

d) DNA sequences comprising a nucleotide sequence, which is degenerate to a nucleotide sequence of c), or parts of said nucleotide sequence,

e) DNA sequences, which represent a derivative, analogue or fragment of a nucleotide sequence of a), b), c) or d).

Another embodiment of the present invention provides a DNA sequence, which codes for a protein having the enzymatic activity of a β-ketoacyl ACP synthase III from Cuphea lanceolata.

Preferably, the latter DNA sequence according to the present invention is selected from the group consisting of

a) DNA sequences comprising a nucleotide sequence, which encodes the amino acid sequence identified in SEQ ID NO: 4 or fragments thereof,

b) DNA sequences comprising the nucleotide sequence identified in SEQ ID NO: 3, or parts thereof,

c) DNA sequences comprising a nucleotide sequence, which hybridises to a complementary strand of the nucleotide sequence of a) or b), or parts of said nucleotide sequence,

d) DNA sequences comprising a nucleotide sequence, which is degenerate to a nucleotide sequence of c), or parts of said nucleotide sequence,

e) DNA sequences, which represent a derivative, analogue or fragment of a nucleotide sequence of a), b), c) or d).

In the context of the present invention the term “hybridisation” means a hybridisation under conventional hybridisation conditions, preferably under stringent conditions, for example as described in Sambrook et al. (1989), Molecular Cloning: A Laboratory Manual, 2. Ed., Cold Spring Harbour Laboratory Press, Cold Spring Harbour, NY.

Plant enzymes having the activity of a β-ketoacyl ACP synthase III (KAS III) possess a highly active regulatory function for controlling the biosynthesis of fatty acids. Based on that knowledge it has now surprisingly been found that by introducing mutations within the region, representing the site which is responsible for the regulatory function of the KAS III, it is possible to increase the content of short and/or middle chain fatty acids in plants or micro-organisms by means of transferring sequences that code for such KAS III mutants. According to the present invention this observation is used to increase the content of short and/or middle chain fatty acids in plants and micro-organisms.

Therefore, in an important aspect of the present invention a DNA sequence is provided which encodes a protein having the enzymatic activity of a β-ketoacyl ACP synthase III, wherein the protein is not controllable by acyl ACPs, especially not inhibited by acyl ACPs.

In a preferred embodiment DNA sequences are provided which, compared to the wild-type sequence of KAS III, are modified in that they have at least one mutation within the region encoding the amino acid sequence motif GNTSAAS (see FIG. 1, in bold).

Especially preferred is a DNA sequence, wherein the mutation results in a substitution of amino acid N by D and/or of amino acid A (first alanine of the motif) by S within the amino acid motif GNTSAAS of KAS III.

In a specific embodiment the present invention provides a DNA sequence, which encodes a protein having the enzymatic activity of a KAS III from Brassica napus, Cuphea lanceolata or Cuphea wrightii, wherein the protein is not controllable by, especially not inhibited by, acyl ACPs.

Particularly, the DNA sequence according to the invention is selected from the group consisting of

a) DNA sequences comprising a nucleotide sequence, which code for the amino acid sequence identified in SEQ ID NO: 6 or fragments thereof,

b) DNA sequences comprising the nucleotide sequence identified in SEQ ID NO: 5 or parts thereof,

c) DNA sequences comprising a nucleotide sequence, which hybridises to a complementary strand of the nucleotide sequence of a) or b), or parts of said nucleotide sequence,

d) DNA sequences comprising a nucleotide sequence, which is degenerate to a nucleotide sequence of c), or parts of said nucleotide sequence,

e) DNA sequences, which represent a derivative, analogue or fragment of a nucleotide sequence of a), b), c) or d).

Finally, the present invention provides chimeric gene constructs, wherein DNA sequences coding for KAS III are under control of regulatory sequences which provide for a specific transcription, by using conventional cloning methods (e.g. Sambrook et al., vide supra). Thus, the present invention further provides a recombinant nucleic acid molecule, which comprises

a) a promoter region,

b) a DNA sequence according to the invention as described above, which is operatively linked to the promoter region, and

c) optionally, regulatory sequences operatively linked thereto, which may act as transcription, termination and/or polyadenylation signals in plant cells.

Alternative embodiments of the present invention provide recombinant nucleic acid molecules, wherein the DNA sequence is oriented in anti-sense.

Preferably the nucleic acid sequence within the recombinant nucleic acid molecule according to the invention is in combination with a promoter active in plants, more preferably in combination with a promoter active in tissues that synthesise and/or store triacylglycerols. Tissues that synthesise and/or store triacylglycerols are primarily seed tissues. But also other plant tissues, such as fruit flesh in oil plants are considered herein as well. Further it may be preferred that the nucleic acid sequence within the

recombinant nucleic acid molecule according to the invention is also present in combination with enhancer sequences, sequences coding for leader peptides and/or other regulatory sequences.

The present invention further provides vectors, which comprise the DNA sequence of the present invention, or the recombinant nucleic acid molecule of the present invention, respectively, both as described above.

A further embodiment of the present invention provides a recombinant protein having the enzymatic activity of a β-ketoacyl ACP synthase III originating from Brassica napus, especially a protein having the amino acid sequence, which is identified in SEQ ID NO:2.

Furthermore, the present invention provides a recombinant protein having the enzymatic activity of a KAS III originating from Cuphea lanceolata, especially a protein having the amino acid sequence, which is identified in SEQ ID NO:4.

A further embodiment of the present invention relates to a recombinant protein having the enzymatic activity of a KAS III, wherein the protein is not controllable by, especially not inhibited by, acyl ACPs. In a specific embodiment the above mentioned protein of the present invention is from Cuphea lanceolata and more preferably has the amino acid sequence identified in SEQ ID NO: 6.

The invention further relates to a method for increasing the content of short chain and/or middle chain fatty acids in plants, which comprises the steps

a) producing a nucleic acid sequence, which codes for a protein having the enzymatic activity of a β-ketoacyl ACP synthase III, wherein the β-ketoacyl ACP synthase III is not regulated by, especially not inhibited by, acyl ACPs, and which comprises at least the following components which are successively arranged in 5′-3′-orientation,

a promoter, which is active in plants, especially in tissues that synthesise and/or store triacylglycerols

at least one nucleic acid sequence, which codes for a protein having the enzymatic activity of a β-ketoacyl ACP synthase III, wherein the β-ketoacyl ACP synthase III is not regulated by, especially not inhibited by, acyl ACPs, or which codes for an active fragment thereof, and

optionally, a termination signal for transcription termination and addition of a poly(A) tail to the corresponding transcript, as well as, optionally, DNA sequences, derived therefrom,

b) transferring the nucleic acid sequence from a) to plant cells, and

c) optionally, regenerating fully transformed plants, and, if desired, propagating the plants.

The invention further relates to methods for increasing the content of short chain and/or middle chain fatty acids in micro-organisms, especially bacteria and algae, which comprises the steps,

a) producing a nucleic acid sequence, which codes for a protein having the enzymatic activity of a β-ketoacyl ACP synthase III, wherein the β-ketoacyl ACP synthase III is not controllable by, especially not inhibited by, acyl ACPs, and which comprises at least the following components which are successively arranged in 5′-3′-orientation,

a promoter, which is active in the respective micro-organism,

at least one nucleic acid sequence, which codes for a protein having the enzymatic activity of a β-ketoacyl ACP synthase III, wherein the β-ketoacyl ACP synthase III is not controllable by, especially not inhibited by, acyl ACPs, or which codes for an active fragment thereof, and

optionally, a termination signal for transcription termination and addition of a poly(A) tail to the corresponding transcript, as well as, optionally, DNA sequences, derived therefrom,

b) transferring the nucleic acid sequences from a) to the respective micro-organism.

In a preferred embodiment the method of the present invention for increasing the content of short chain and/or middle chain fatty acids in plants or micro-organisms, respectively, comprises the following steps b)-c) for micro-organisms or b)-d) for plants, respectively, after the above mentioned step a),

b) inactivating the acyl ACP binding site of the β-ketoacyl ACP synthase III by in vivo mutation,

c) transferring the nucleic acid sequences from a) or b), and

d) as far as the nucleic acid sequences in step c) have been transferred to plant cells, optionally regenerating fully transformed plants, and, if desired, propagating the plants.

A further subject-matter of the present invention are transgenic plants and micro-organisms, which contain a DNA sequence of the present invention as mentioned above or a recombinant nucleic acid molecule of the present invention as described above.

Particularly the invention relates to transgenic plants, plant cells and micro-organisms, which contain a nucleic acid sequence coding for a protein having the activity of a β-ketoacyl ACP synthase III, wherein the β-ketoacyl ACP synthase III is not regulated by, especially not inhibited by, acyl ACPs. Studies of the influence of acyl ACPs of different chain lengths on the activity of KAS III from Cuphea demonstrated that the KAS III enzymes from Cuphea are involved in the regulation of the biosynthesis of middle chain fatty acids via a strong feedback inhibition, which is effected by the middle chain acyl ACP end products that are synthesised in the plastids of the corresponding seeds. Our kinetic studies with recombinant KAS III from Cuphea wrightii further demonstrated that there are different binding sites for the inhibitory C₁₂-ACP and the substrates acetyl CoA and malonyl ACP.

Therefore in a preferred embodiment the inventive plants and micro-organisms contain a nucleic acid sequence coding for a KAS III mutant, wherein the regulatory function at the binding site of the acyl ACPs is knocked out by one or several mutations, but wherein the catalytic activity in the condensation reaction of acetyl CoA and malonyl ACP is maintained. In the case of KAS III mutants from Cuphea, this results in an uninhibited synthesis of acyl ACPs, which themselves inhibit the enzyme KAS IV, being responsible for the synthesis of middle chain fatty acids, and the enzyme KAS II, being responsible for the synthesis of long chain fatty acids. In this way in Cuphea the synthesis is shifted towards short chain fatty acids, especially C₄-C₈ fatty acids, whereas in rape-seed the synthesis is shifted towards middle chain fatty acids, especially C₆-C₁₀ fatty acids.

In a further preferred embodiment the plants and micro-organisms according to the invention contain nucleic acid sequences which, compared to the wild-type sequence of KAS III from C. wrightii (Slabaugh et al. (1995): Plant Physiol. 108, 343-344), are altered by at least one mutation within the region that encodes the amino acid sequence motif G³⁵⁷NTSAAS³⁶³. As will be further explained in detail below, the amino acid sequence motif G³⁵⁷NTSAAS³⁶³ from C. wrightii is a motif conserved in KAS III enzymes. In the KAS III from C. wrightii this motif GNTSAAS is between the amino acids 357 and 363, calculated from the start of the pre-sequence, which codes for a pre-KAS III including a leader peptide, which is responsible for the transport into the plastids. With respect to mature KAS III protein from C. wrightii the amino acid motif is localised between amino acid 290 and amino acid 296. The precise position of the amino acid motif GNTSAAS according to the invention in KAS III enzymes from various organisms can be taken from FIG. 1.

Since the position of the motif GNTSAAS varies in the various KAS III, unless the KAS enzyme from C. wrightii is explicitly referred to, it will hereinafter be generally referred to the motif GNTSAAS, without giving details of particular amino acid positions (which may be taken from FIG. 1 and from additional sequence alignments which may be easily drawn up by one skilled in the art).

In the framework of the present invention it could be shown by kinetic studies, especially by aid of the mutants Asn³⁵⁸Asp (N291D in the mature protein) and Ala³⁶¹Ser (A294S in the mature protein) of the recombinant C. wrightii KAS III that due to these mutations at the regulatory binding site these enzymes are no longer inhibited by acyl ACPs, but maintain their full catalytic activity. It could be demonstrated that in plants of the present invention, which are transformed with the KAS III mutant Asn³⁵⁸Asp, the synthesis of C₄-ACPs is not only considerably stimulated by the mutant, but also, for example, in Cuphea lanceolata the synthesis of C₄-C₆ acyl ACPs is increased by 50% at the expense of middle chain acyl ACPs. In transgenic rape-seed expressing the above mentioned KAS III mutants, the synthesis of middle chain acyl ACPs (C₆-C₁₀) was also increased by more than 50% at the expense of long chain acyl ACPs.

In a preferred embodiment, for use in the method of the present invention for increasing the content of short and/or middle chain fatty acids, the KAS III sequences are expressed in plant cells under control of seed-specific regulatory elements, especially promoters. Thus, in a preferred embodiment the above mentioned DNA sequences are in combination with promoters, which are especially active in tissues that synthesise or store triacylglycerols, such as embryonic tissue or fruit flesh in oil plants. Examples for such promoters are the USP promoter (Bäumlein et al. 1991, Mol. Gen. Genet. 225:459-467), the Hordein promoter (Brandt et al. 1985, Carlsberg Res. Commun. 50:333-345) and the Napin promoter, the ACP promoter as well as the FatB3- and FatB4 promoters, which are well known to a person skilled in the art of plant molecular biology.

For the use in the inventive method the nucleic acid sequences may optionally be supplemented by enhancer sequences or other regulatory sequences. The regulatory sequences comprise, for example, also leader sequences which provide for the transport of the gene product to a specific compartment. Signal sequences deserving particular mention are those that direct the gene product to the site of fatty acid synthesis in the plant, that is the plastids. If the chloroplast transformation is utilised, the nucleic acid sequence coding for the KAS III is directly incorporated into the plastid genome so that usually no corresponding leader sequences or leader peptides are required.

The present invention also relates to nucleic acid molecules, which contain the above mentioned nucleic acid sequences or parts thereof, i.e. also vectors, especially plasmids, cosmids, viruses, bacteriophages and other vectors, which are commonly used in genetic engineering, which, if desired, may be used for the transfer of the above mentioned nucleic acid molecules to plants or plant cells.

Plants which are transformed according to the present invention and in which as a result of the transformation an altered amount of fatty acids is synthesised, may in principle be any plant. Preferably, it is a monocotyledonous or dicotyledonous crop plant and more preferably an oil plant. In particular, rape seed, sunflower, soybean, peanut, coconut, turnip seed, cotton and oil palm trees are mentioned as examples. Further plants which may serve for the production of fatty acids and lipids, or may be useful as foodstuffs with an increased content of fatty acids, are flax, poppy, olive, cocoa, maize, almond, sesame, mustard and castor oil plant.

The invention further relates to propagating material of the plants according to the invention, for example seeds, fruits, cuttings, tubers, rhizomes and the like, as well as parts of these plants such as protoplasts, plant cells and calli.

The micro-organisms which are transformed in the present invention and in which as a result an altered amount of fatty acids is synthesised may in principle be any micro-organism. Bacteria or algae are preferred.

In a preferred embodiment the transgenic plants and micro-organisms contain a nucleic acid sequence, which codes for a protein having the activity of a β-ketoacyl ACP synthase III from Brassica napus, Cuphea lanceolata or Cuphea wrigthii, wherein the β-ketoacyl ACP synthase III is not regulated by, especially not inhibited by, acyl ACPs.

The KAS III nucleic acid molecules, which are useful in the present invention, also comprise fragments, derivatives and allelic variants of the above described DNA sequences encoding a KAS III or a biologically, i.e. enzymatically, active fragment thereof. Fragments are to be understood as parts of the nucleic acid molecules which are long enough to encode a polypeptide or a protein having the enzymatic activity of a KAS III or a comparable enzymatic activity. The term derivative means in this context that the sequences of these molecules differ from the sequences of the above mentioned nucleic acid molecules at one or more positions and have a high degree of homology to these sequences. Homology in this context means a sequence identity of at least 80%, 90%, and 92%, especially an identity of at least 94% and 96%, preferably of more than 98% and more preferably of more than 99%, or that the homologous sequence hybridises to the aforementioned KAS III sequences, under stringent conditions, as they are familiar to the person skilled in the art. The variation to the above described nucleic acid molecules may be caused by deletion, addition, substitution, insertion or recombination. Homology further means that there is a functional and/or structural equivalence between the respective nucleic acid molecules or the proteins encoded by them.

The nucleic acid molecules which are homologous to the above mentioned molecules and which represent derivatives of these molecules are usually variations of these molecules, which represent modifications that exhibit the same biological function. These variations may be naturally occurring variations, for example sequences from other organisms, or mutations, wherein these modifications may have occurred in a natural way or may have been introduced by targeted mutagenesis. Further the variations may be synthetic sequences. The allelic variants may be naturally occurring or synthetic variants or variants created by recombinant DNA techniques.

Conventionally the KAS III proteins which are encoded by the different variants of the nucleic acid sequences which are useful in the present invention have certain common characteristics. These are, for example enzyme activity, molecular weight, immunological reactivity, conformation and the like. Further common characteristics may be physical properties such as gel electrophoretic mobility, chromatographic behaviour, sedimentation coefficients, solubility, spectroscopic properties, stability, pH optimum, temperature optimum and the like. Furthermore the products of the reactions catalysed by the KAS III enzymes may have common or similar features.

Various methods can be used for the production of the plants according to the present invention. On the one hand plants or plant cells may be modified by conventional transformation techniques used in genetic engineering in such a way that the novel nucleic acid molecules are integrated into the plant genome, i.e. that stable transformants are created. On the other hand, an above-mentioned nucleic acid molecule, whose presence and possible expression in the plant cell effects a change in the fatty acid content, may be contained as a self-replicating system within the plant or plant cell.

In order to prepare the introduction of foreign genes in higher plants a bulk of cloning vectors are available which contain replicating signals for E. coli and a marker gene for selecting transformed bacterial cells. Examples for such vectors are pBR322, pUC series, M13mp series, pACYC184, pBlueSfi and the like. The desired sequence may be introduced in the vector at a suitable restriction site. The resulting plasmid is then used for the transformation of E. coli cells. Transformed E. coli cells are cultivated in a suitable medium and then harvested and lysed, and the plasmid is recovered. In order to characterise the produced plasmid DNA in general restriction analysis, gel electrophoresis and further biochemical and molecular biological methods are used as analytic method. After each manipulation step the plasmid DNA may be cleaved and the obtained DNA fragments may be linked to other DNA sequences.

Several known techniques are available for introducing DNA in a plant host cell, and the person skilled in the art will not have any difficulties in selecting a suitable method. These techniques comprise the transformation of plant cells with T DNA by use of Agrobacterium tumefaciens or Agrobacterium rhizogenes as the transforming agent, the fusion of protoplasts, the direct gene transfer of isolated DNA into protoplasts, the electroporation of DNA, the introduction of DNA by means of the biolistic method as well as further possibilities.

During the injection and electroporation of DNA into plant cells no specific requirements for the used plasmids are necessary per se. The same is true for the direct gene transfer. Plain plasmids such as pUC and pBlueScript derivatives may be used. The presence of a selectable marker gene is necessary, if entire plants are to be regenerated from such transformed cells. The person skilled in the art is familiar with these gene selection markers and he will not have any problems in selecting a suitable marker.

Further DNA sequences may be required depending on the introduction method for desired genes into the plant cell. If, for example, the Ti or Ri plasmid is used for the transformation of the plant cell, at least the right border, however more often both, the right and the left border of the T DNA in the Ti or Ri plasmid, has to be linked as flanking region to the genes to be introduced.

If agrobacteria are used for the transformation, the DNA to be introduced has to be cloned into special plasmids, either into an intermediate or into a binary vector. Intermediate vectors may be integrated into the Ti or Ri plasmid of the agrobacteria by homologous recombination due to sequences which are homologous to sequences in the T DNA. This also contains the vir region which is required for T DNA transfer. Intermediate vectors are not able to replicate in agrobacteria. With the aid of a helper plasmid, the intermediate vector may be transferred to Agrobacterium tumefaciens (conjugation). Binary vectors are able to replicate in E. coli as well as in agrobacteria. They contain a selection marker gene, and a linker or polylinker framed by the right and left T DNA border region. They may be transformed directly into agrobacteria. The agrobacterial host cell should contain a plasmid with a vir region. The vir region is required for the transfer of the T DNA into the plant cell. Additional T DNA may be present. Such a transformed agrobacterial cell is used for the transformation of plant cells.

The use of T DNA for the transformation of plant cells has been studied intensively, and has been sufficiently described in generally known reviews and plant transformation manuals. For transfer of the DNA into the plant cell, plant explantates may be cultivated for this purpose together with Agrobacterium tumefaciens or Agrobacterium rhizogenes. From the infected plant material (e.g. leaf pieces, stem segments, roots, but also protoplasts or suspension-cultivated plant cells) whole plants may be regenerated in a suitable medium which may contain antibiotics or biocides for selection of transformed cells. Regeneration of plants may take place according to conventional regeneration methods by use of known growth media. The resulting plants may be analysed for the presence of the introduced DNA. Other possibilities for the introduction of foreign DNA by use of biolistic methods or protoplast transformation are well known, as well as have been described extensively.

Once the introduced DNA has been integrated into the plant cell genome it is generally stable there and is maintained in the progeny of the originally transformed cell as well. Normally it contains a selection marker which endows the transformed plant cells with resistance to a biocide or an antibiotic, such as kanamycin, G418, bleomycin, hygromycin, methotrexate, glyphosate, streptomycin, sulfonylurea, gentamycin or phosphinotricin and others. The individually selected marker should therefore allow the selection of transformed cells from cells lacking the introduced DNA. For example nutritive markers, screening markers (such as GFP, green fluorescent protein) moreover are useful as alternative markers. Naturally it could also be done without any selection marker, although this would involve a quite high screening expenditure.

Within the plant the transformed cells grow by a normal way. The resulting plants may be cultivated normally, and may be crossbred with plants having the same transformed hereditary disposition or other predispositions. The resulting hybrids will have the pertinent phenotype characteristics. Seeds may be obtained from the plant cells.

Two or more generations should be generated to ensure that the phenotypic feature is stably maintained and inherited. Additionally, seeds should be harvested to verify the maintenance of the respective phenotype or other features.

Transgenic lineages, which are homozygous for the novel nucleic acid molecules, may be determined by usual methods as well, and their phenotypic behaviour may be analysed in view of a modified fatty acid content, and may be compared to the behaviour of hemizygous lineages.

For detecting the expression of the proteins having KAS III activity which cannot be regulated, conventional molecular biological and biochemical methods may be used. These techniques are well known to a person skilled in the art and he won't have any problems in selecting a suitable detection method, such as a Northern blot analysis for detecting KAS-specific RNA or for determining the amount of accumulation of KAS-specific RNA, respectively, a Southern blot analysis for identifying DNA sequences encoding KAS III or a Western blot analysis for detecting the KAS III protein encoded by the DNA sequences according to the present invention. Detection of the enzymatic activity of the KAS III may be determined by means of the fatty acid pattern or an enzyme assay which for example are described in the subsequent examples.

The invention is based on the successful production and characterisation of novel KAS III sequences and KAS III mutants and the classification of concrete substrate specificities which has been successfully done for the first time, as well as the elucidation of the KAS III regulation mechanisms, which will be described in the following examples.

The following examples are intended to illustrate the present invention.

### EXAMPLES

### Example 1

### Site-Specific Mutagenesis of Cuphea wrightii KAS IIIa cDNA

Standard DNA engineering techniques have been performed as described in Sambrook et al. (J. Sambrook, E. F. Fritsch, T. Maniatis (1989), Molecular Cloning: A Laboratory Manual, 2^{nd} edition, Cold Spring Harbor, N.Y.). The starter plasmid for the generation of the KAS III mutants was cwKAS IIIa cDNA (cwKAS=Cuphea wrightii KAS), which was cloned into the expression vector pET 15b (Novagen, MA, USA) via the NdeI and XhoI restriction sites. The mutated DNA was created by use of the PCR based overlap extension technique (R. Higuchi, B. Krummel, R. K. Seiki (1988), Nucl. Acids Res. 16, p. 7351-7367). The sequences of the oligonucleotides used as primers for the PCR reaction are shown in Table 1.

TABLE 1

| Primer | Sequence^{a} |
|---|---|
| 5′ flanking primer | 5′-TGGAAAGGCCGGCCTTAATG-3′ |
| Fse5 | |
| 3′ flanking primer | 5′-CTCGAGTTATCCCCACCTGAT-3′ |
| Xho3 | |
| 5′ mutation primer | 5′-AACTACGGGGACACTAGTGC-3′ |
| Asn³⁵⁸Asp-5 | |
| 3′ mutation primer | 5′-GCACTAGTGTCCCCGTAGTT-3′ |
| Asn³⁵⁸Asp-3 | |
| 5′ mutation primer | 5′-AACACTAGTTCGGCATCCATT-3′ |
| Ala³⁶¹Ser-5 | |
| 3′ mutation primer | 5′-AATGGATGCCGAACTAGTGTT-3′ |
| Ala³⁶¹Ser-3 | |
| 5′ mutation primer | 5′-CACTAGTGCGCCATCCATTC-3′ |
| Ala³⁶²Pro-5 | |
| 3′ mutation primer | 5′-GAATGGATGGCGCACTAGTG-3′ |
| Ala³⁶²Pro-3 | |
| 5′ mutation primer | 5′-GCAAACTACGCGGCATCCA-3′ |
| deletion-5 | |
| 3′ mutation primer | 5′-TGGATGCCGCGTAGTTGGC-3′ |
| deletion-3 | |

^{a}the mutagenised codons are underlined.

First, the target site (base pairs 734-1014), at which mutations are to be introduced, of the cDNA of the presumably mature cwKAS IIIa (starting from the codon encoding the amino acid G68 to the stop codon) was cloned into the pGEM-T Easy vector (Promega, Heidelberg) as a mutation cassette. This construct is hereinafter referred to as K3KM-pGEMT. The mutation cassette was constructed by PCR reaction by the use of the primers Fse5 and Xho3, resulting in the amplification of a 280 base pair fragment. The PCR conditions were as follows: initial denaturation at 95° C. for 30 seconds, followed by 25 cycles of annealing at 55° C. for 30 seconds, elongation at 72° C. for 1 minute and denaturation at 95° C. for 30 seconds. The last step of DNA synthesis was performed at 72° C. for a period of 5 minutes. The amplification of the DNA fragment was performed with 50 pmol of each primer, 1.3 U proof-reading Pfu polymerase, 2 ng pET15b-cwKASIIIa plasmid as template and 200 μM dNTPs in a total volume of 50 μl. The resulting DNA fragment was introduced into the pGEM-T Easy Sequencing Vector (Promega, Heidelberg, Germany) by ligation, following the manufacturer's protocol. The entire sequence of the mutation cassette was confirmed by DNA sequencing.

Then, three point-mutated KAS IIIs were created by the substitution of asparagine³⁵⁸ by aspartate, alanine³⁶¹ by serine and alanine³⁶² by proline. The amplification of the overlapping DNA fragments of the KAS III mutants was performed by two separate reactions, which each contained 2 ng K3MK-pGEMT plasmid as template, 2.5 U proof-reading Pfu polymerase, 200 μM dNTPs and 50 pmol of each of the primers in a total volume of 50 μl. Each reaction mix contained a flanking primer (Fse5 or Xho3) and the corresponding mutation primer. The PCR conditions were as follows: denaturation at 94° C. for 2 minutes, followed by 30 cycles at 94° C. for 30 seconds, 55° C. for one minute and 72° C. for one minute and a final elongation step at 72° C. for 10 minutes. For creating the full length of the mutagenised DNAs 2.0 ng gel-purified overlapping DNA fragments were used in a second reaction, with 50 pmol of each of the flanking primers Fse5 and Xho3, 200 μM dNTPs and 2.5 U Pfu polymerase in a total reaction volume of 50 μl. The PCR conditions were as follows: denaturation at 94° C. for 2 minutes, followed by 30 cycles at 94° C. for 30 seconds, 50° C. for one minute and 72° C. for one minute and a final elongation step at 72° C. for 10 minutes. The sequence of the mutant constructs was confirmed by DNA sequencing after ligation into the pGEM-T Easy Sequencing vector. For protein expression the cDNAs of the KAS IIIa mutants were subcloned into the FseI and XhoI restriction sites of the pET15b-cwKAS IIIa plasmid.

The deletion mutant of KAS III was created by complete deletion of the amino acid motif Gly³⁵⁷Asn³⁵⁸Thr³⁵⁹Ser³⁶⁰ of the wild-type KAS IIIa. The amplification of the overlapping DNA fragments was performed by two separate reactions with 2 ng K3MK-pGEMT plasmid as template, 2.5 U proof-reading Pfu polymerase, 200 μM dNTPs and 50 pmol of each of the primers in a total volume of 50 μl. In order to amplify the mutagenised overlapping DNA fragments the primer pairs Fse5/Del3 and Xho3/Del5 were used. The conditions for the PCR were as follows: denaturation at 94° C. for 2 minutes, followed by 30 cycles at 94° C. for 30 seconds, 55° C. for one minute and 72° C. for one minute and a final elongation step at 72° C. for 10 minutes. The complete mutant DNA fragment was created in a second reaction with 2.0 ng of the gel-purified overlapping DNA fragments with 50 pmol of each of the primers Fse5 and Xho3, 200 μM dNTPs and 2.5 U Pfu polymerase in a total reaction volume of 50 μl. The resulting DNA fragment was sequenced and subcloned in the same way, as described above for the other mutant constructs.

### Example 2

### Expression and Purification of Recombinant Wild-Type KAS IIIa and KAS IIIa Mutants

Wild-type KAS III and KAS IIIa mutants were expressed with a His₆-tag at the N-terminus in E. coli strain BL21(DE3)pLys (Novagen, Madison, USA) and purified by nickel affinity chromatography. Purity of the synthesised KAS IIIs was evaluated by SDS-PAGE. The KAS III concentration was determined by the method of Bradford (M. M. Bradford (1976), Anal. Biochem. 72, p. 248-254).

### Example 3

### Enzyme Assays and Inhibition Studies

The activity of KAS IIIa was analysed by the incorporation of radioactive acetate from [1-¹⁴C] acetyl CoA into acetoacetyl ACP (Brück et al. (1996), Planta 198, p. 271-278). The reaction mix (50 μl) contained 100 mM sodium phosphate, pH 7.6, 10 μM [1-¹⁴C] acetyl CoA, 20 μM malonyl ACP and 2 ng of the recombinant KAS IIIa or the KAS IIIa mutant, respectively. The reaction was initiated by adding the enzyme and was performed for a period of 5 minutes at 30° C.

In the inhibition studies of the wild-type KAS IIIa the used conditions provided for saturation with respect to the substrates acetyl CoA and malonyl CoA. As regards the inhibitors non-radioactive acyl ACPs (C2-C16) or acyl CoAs (C3-C12) were added at varying concentrations. The resulting data were analysed by the method of Lineweaver-Burk. The inhibition studies of the KAS IIIa mutants with acyl ACPs were performed with 10 μM non-radioactive dodecanoyl ACP (see FIG. 3).

Table 2 shows the results for the inhibition of wild-type KAS IIIa by acyl ACPs and acyl CoAs. The KAS III activity was measured as described above in the presence of varying concentrations of acyl ACP (5-25 μM) or of acyl CoA (10-50 μM).

TABLE 2

| | | |
|---|---|---|
| chain length | acyl ACPs | acyl CoAs |

| carbon atoms | Kᵢ [μM] | |
|---|---|---|
| 2 | 2.16 ± 0.4 | not determined |
| 3 | not determined | 20.80 ± 0.51 |
| 4 | 2.9 ± 0.3 | 91.9 ± 0.9 |
| 6 | 8.0 ± 0.2 | 42.7 ± 0.3 |
| 8 | 4.1 ± 0.3 | 24.4 ± 0.6 |
| 10 | 4.1 ± 0.4 | 12.5 ± 0.6 |
| 12 | 0.4 ± 0.1 | 20.0 ± 0.7 |
| 14 | 4.0 ± 0.1 | not determined |
| 16 | 1.3 ± 0.1 | not determined |

### EXAMPLE 4

### Synthes8is of the Acyl ACPs in Plant Extracts Supplemented with the Not Controllable KAS IIIa mutant Asn³⁵⁸Asp

For supplementation experiments a FAS preparation from C. lanceolata seeds was obtained from cell-free extracts by ammonium sulfate precipitation (saturation from 0 to 65%) (Brück et al., supra). The FAS preparation from seeds of rapeseed was obtained as described in MacKintosh et al. (1989, BBA 1002, 114-124). All preparations were stored at −70° C. Prior to use an aliquot of the thawed preparation was dissolved in 1 ml 100 mM sodium phosphate (pH 7.6) and centrifuged (10,000×g, 5 minutes, 4° C.) to eliminate any insoluble material.

The influence of supplemented KAS IIIa mutants on the acyl ACP pattern in preparations from C. lanceolata seeds of rapeseed was subsequently analysed by means of the incorporation of [1-¹⁴C] acetate from [1-¹⁴ C] acetyl CoA in acyl ACPs. The assays were performed as described in Schütt et al. (Planta 205 (1998), p. 263-268). The reaction mix (200 μl) contained 100 mM sodium phosphate (pH 7.6), 10 μM [1-¹⁴C] acetyl CoA, 20 μM malonyl CoA, 10 μm ACP from E. coli, 1 mM NADH, 2 mM NADPH, 2 mM DTT, FAS preparation (0.205 μg protein per μl reaction mix) and affinity-purified KAS IIIa mutant Asn³⁵⁸Asp in an end concentration of 7.11 ng protein per μl reaction mix. The control reactions were performed by addition of wild-type KAS IIIa instead of the Asn³⁵⁸Asp mutant and without enzyme supplemention. Additional control reactions were performed by addition of 10 μM decanoyl ACP to the reaction mixes. Samples (50 μl) were collected within 30 minutes at different time periods, and the reaction was stopped by precipitation of the acyl ACPs with trichloroacetic acid at an end concentration of 10 vol.-%. The precipitated acyl ACPs were washed, as described in Brück et al., vide supra, dissolved in 18.7 μl MES (pH 6.8) and separated by use of 2.5 M and 5.0 M urea PAGE as described in Post-Beittenmiller et al. (1991, J. Biol. Chem. 266, 1858-1865), transferred to Immobilon P (Millipore, Eschborn, Germany) and visualised by auto-radiography as described in Brück et al. (1996, vide supra) (see FIG. 4). The elongation products were quantified densitometrically by use of an Ultroscan XL-apparatus (Pharmacia, Freiburg, Germany).

Table 3 shows the total enrichment of FAS products by the preparations from C. lanceolata and rapeseed as a function of the added enzyme. The synthesis of FAS products was measured, as mentioned above, by the incorporation of [1-¹⁴C] acetate and by use of a scintillation counter.

TABLE 3

total amount of FAS products (based on pmol of incorporated acetate)

| FAS preparation | FAS | FAS + wtKAS III | FAS + Asn³⁵⁸Asp |
|---|---|---|---|
| C. lanceolata seeds | 158.0 ± 8.6 | not determined | 184.4 ± 4.7 |
| rapeseed seeds | 82.5 ± 7.3 | 85.1 ± 4.8 | 105.4 ± 5.1 |

The resulting ACP pattern is shown in Table 4. In Table 4 as well as in FIG. 4 the acyl groups are defined by the number of carbon atoms: the number of double bonds.

TABLE 4

| | acyl ACP (mol %) | | | | | | | |
|---|---|---|---|---|---|---|---|---|
| FAS preparation | 4:0 | 6:0 | 8:0 | 10:0 | 12:0 | 14:0 | 16:0 | 18:0 |
| C. lanceolata seeds | 25.1 ± 3.1 | 18.4 ± 4.2 | 25.5 ± 4.9 | 14.6 ± 3.9 | 9.3 ± 1.9 | 2.0 ± 0.6 | 2.7 ± 0.4 | 2.4 ± 0.9 |
| C. lanceolata seeds + | 32.8 ± 4.7 | 48.6 ± 6.1 | 20.3 ± 4.1 | 2.3 ± 0.6 | 2.0 ± 0.5 | n.d. | n.d. | n.d. |
| Asn³⁵⁸Asp | | | | | | | | |
| rapeseeds | 13.2 ± 2.7 | 13.4 ± 3.4 | 19.1 ± 2.2 | 11.3 ± 1.8 | 11.7 ± 1.7 | 11.1 ± 2.3 | 14.4 ± 3.6 | 5.8 ± 1.1 |
| rapeseeds + wtKASIII | 14.8 ± 1.5 | 14.5 ± 2.8 | 18.1 ± 2.8 | 11.9 ± 2.3 | 12.3 ± 2.3 | 11.6 ± 2.7 | 12.6 ± 4.1 | 4.2 ± 0.4 |
| rapeseeds + Asn³⁵⁸Asp | 17.3 ± 2.3 | 19.6 ± 3.1 | 23.7 ± 4.2 | 12.8 ± 3.6 | 11.0 ± 3.2 | 7.5 ± 1.7 | 6.1 ± 1.8 | 2.0 ± 0.2 |

n.d. = not determined

Kinetic studies with recombinant KAS IIIa from C. wrightii revealed that the inhibition of this condensing enzyme by dodecanoyl ACP is not competitive with respect to acetyl CoA or malonyl CoA (see FIG. 2). This gives a hint that there are other binding sites for the inhibitory C₁₂ ACP than for acetyl CoA and malonyl CoA. In order to identify the amino acids involved in the regulatory region of KAS III, an amino acid sequence alignment was performed from known KAS III sequences from plants, red alga (Porphyra umbilicalis and E. coli), as well as from those of the present invention, (for references see below in the context with FIG. 1). Comparative analysis of the primary structures shown in FIG. 1 demonstrates the existence of a highly conserved region G³⁵⁷NTSAAS³⁶³ at the C-terminus.

Deletion of the entire peptide Gly³⁵⁷Asn³⁵⁸Thr³⁵⁹Ser³⁶⁰ resulted in the complete loss of the enzymatic activity. Studies on the secondary structure of this mutant demonstrated that the loss of activity was rather due to an alteration of the entire structure as compared to the active conformation of the wild-type KAS III than to catalytic properties of this motif.

In order to analyse the contribution of certain amino acids to the regulatory function three mutants Asn³⁵⁸Asp, Ala³⁶¹Ser and Ala³⁶²Pro were created. Secondary structure analysis of these mutants using CD spectroscopy demonstrated that the spectra of the Asn³⁵8Asp and Ala³⁶¹ Ser mutants were essentially the same that of wild-type KAS IIIa, whereas the secondary structure of the Ala³⁶²Pro mutant was altered, the latter resulting in a decreased condensing activity (see FIG. 5).

The inhibition of the Asn³⁵⁸Asp and Ala³⁶¹Ser mutants by dodecanoyl ACP demonstrated that the mutants were almost not at all inhibited by this acyl ACP (see FIG. 2). The mutants maintained about 85% of their initial activity in the presence of a saturation concentration (10 μM) of dodecanoyl ACP.

The kinetic data show that the recombinant wild-type KAS IIIa has an individual binding site for the regulatory acyl ACP, wherein the bonding does not occur covalently. The finding that the Asn³⁵⁸Asp and Ala³⁶¹Ser mutants are not inhibited by acyl ACPs is likely to be a consequence of a change in charge and/or polarity of the side chains of the corresponding amino acids, hindering the docking of the acyl ACP.

The results obtained with the FAS preparations from extracts of C. lanceolata seeds and rapeseeds revealed differences in the total enrichment of FAS products as a function of the supplemented enzyme (Table 3). Supplementation with the Asn³⁵⁸Asp mutant led to a 1.2-fold enrichment of FAS products in extracts of C. lanceolata and rapeseeds. In order to prove whether this effect is due to the KAS III activity, i.e. the production of butyryl ACP, or to the knockout of a regulatory site of the FAS enzyme complex, the rapeseed extracts were supplemented with the same amount of wild-type KAS III as in the case of the mutant. In these experiments, no significant meaningful differences could be observed in the total amount of FAS products compared to that without supplementation. This indicates that the Asn³⁵⁸Asp mutant has an influence on the total synthesis of acyl ACP.

Addition of the KAS IIIa mutant in excess to extracts from C. lanceolata and rapeseeds demonstrated that the mutant—as was expected—not only considerably stimulated the synthesis of C₄ACP in both extracts, but also led to an increase of 50% in the synthesis of C₄-C₆ acyl ACPs in the C. lanceolata extract (at the expense of middle chain acyl ACPs, especially C₁₀ and C₁₂), and to an increase of also more than 50% in the synthesis of middle chain acyl ACPs (C₆-C₁₀) in the rapeseed extract, here at the expense of long chain acyl ACPs, especially of C₁₄ to C₁₈ acyl ACPs (see Table 4).

Such modifications in the acyl ACP composition reflect the modifications in the regulation and control of the fatty acid biosynthesis. These modifications are probably caused by a change in the stoichiometric relation of some acyl ACPs, which itself could influence other condensing enzymes of the FAS enzyme complex.

### Example 5

### Cloning of KAS III cDNAs from Cuphea lanceolata and Brassica napus

Total RNA was isolated from embryos of developing seeds of Cuphea lanceolata and Brassica napus as described in Voetz et al. (1994, Plant Physiol. 106: 785-786). The mRNA was extracted by the use of oligo-dT cellulose (Qiagen, Hilden, Germany) according to the manufacturer's protocol. The cDNA sequences were obtained from mRNA preparations by RT-PCR with NotI-dT₁₈-primers (see Table 5) using the “first strand synthesis” kit (Pharmacia, Freiburg, Germany). The degenerate oligonucleotides 5a/3a and 5b/3b, respectively, based on conserved regions of the KAS III encoding genes (see Table 5) were used as primers to amplify overlapping cDNA fragments by PCR (see FIG. 1).

The PCR reaction mix contained 200 μM dNTPs, 100 pMol of each of the primers, 1.5 μl of the cDNA pool, 2.5 U Taq DNA polymerase within a total volume of 50 μl. The following temperature program was used: initial denaturation for 3 minutes at 94° C., followed by 35 cycles of denaturation for 1 minute at 94° C., annealing for 1 minute at 52° C. and elongation for 1 minute at 72° C., followed by a final elongation step for 10 minutes at 72° C.

The KAS III DNA sequence of the resulting overlapping 923 bp and 1013 bp fragments was verified by automated DNA sequencing and alignment of the deduced amino acid sequences with known KAS III protein sequences.

Sequence information about the remaining and still unknown 3′-region were determined by 3′-RACE (Rapid Amplification of CDNA Ends) with the NotI-dT₁₈-adapter primer and clKAS III sequence-specific internal primers, which were deduced from sequence information obtained from the overlapping CDNA fragments.

The PCR conditions for 3′-RACE were as follows: 200 μM dNTPs, 40 pMol of the sequence-specific primer C1-3′-RACE, 80 pMol NotI-dT₁₈-adapter primer, 5 μl cDNA pool, 5 U Taq DNA polymerase within a final volume of 50 μl. The temperature program was as follows: initial denaturation for 3 minutes at 94° C., followed by 35 cycles of denaturation for 1 minute at 94° C., annealing for 2 minutes at 55° C. and elongation for 2 minutes at 72° C., followed by a final elongation step for 10 minutes at 72° C. The resulting fragment was cloned into a sequencing vector and sequenced by automated DNA sequencing.

Furthermore, a KAS III-cDNA from rapeseed (Brassica napus) was cloned using the same strategy as described above for Cuphea lanceolata and the same degenerate primer pairs (5a/3a and 5b/3b) for the amplification of the overlapping cDNA fragments from a rapeseed cDNA pool, with the difference that a rapeseed-specific primer (Bn-3′-RACE) was used for the 3′-RACE-PCR.

According to the sequence information obtained from the overlapping fragments and the 3′-RACE fragments, cDNAs were determined for clKAS III (see SEQ ID NO: 3) and bnKAS III (see SEQ ID NO: 1), which theoretically are full length clones including the start and stop codon. The cDNAs encode a polypeptide of 402 amino acids in the case of clKAS III (see SEQ ID NO: 4) and 404 amino acids in the case of bnKAS III (see SEQ ID NO: 2).

For heterologous expression of clKAS III and bnKAS III in an E. coli pET15b vector system and additional in vitro experiments with the recombinant enzyme the cDNA encoding the mature protein (the start of the mature protein was defined on the basis of a sequence alignment with KAS III from E. coli and P. umbilicalis, see FIG. 1) was amplified by PCR, which was accompanied by the introduction of 5′-NdeI and 3′-XhoI restriction sites for subcloning.

The PCR reaction mix contained 200 μM dNTPs, primer pairs Cl 5′-NdeI/Cl 3′-XhoI and Bn 5′-NdeI/Bn 3′-XhoI (50 pMol each), 2 μl of the cDNA pool, 2.5 U proof-reading Pfu DNA polymerase within a final volume of 50 μl. The following temperature program was used: initial denaturation for 3 minutes at 94° C., followed by 35 cycles of denaturation for 1 minute at 95° C., annealing for 1 minute at 55° C. and elongation for 2 minutes at 72° C., followed by a final elongation step of 10 minutes at 72° C.

DNA sequencing of the resulting 1023 bp (clKAS III) and 1026 bp (bnKAS III) PCR products demonstrated that they are identical to the corresponding sequences of the overlapping DNA fragments as described above.

### Example 6

### Cloning and Mutagenesis of the clKAS III cDNA

For the production of vector constructs for the transformation of plants with mutagenised clKAS III two “precursor” vector constructs, namely a) the full length of the wildtype cDNA encoding the pre-sequence and the mature protein in a reading frame and b) the corresponding full length site-specifically mutagenised cDNA, served as starting material.

a) Construction of the clKAS III cDNA in Full Length Including the Pre-Sequence

As the pre-peptide is required for the correct transport of the clKAS III into plastids, it had to be integrated into vector constructs used for the transformation of plants with clKAS III. For this purpose a “chimeric” clKAS III gene including the clKAS III pre-sequence was created by way of precise gene fusion on the basis of overlapping PCR according to Yon and Fried (1989, Nucleic Acid Research 17: 4895).

Overlapping cDNA fragments were amplified in two separate reactions, wherein the PCR conditions were as follows: 200 μM dNTPs, primer pairs clprae-5/cloverl-3 and cloverl-5/clctrm-3, respectively (50 pMol each) (see Table 5), 2 ng template (the above-described 1011 bp DNA fragment comprising the clKAS III pre-sequence and the 1009 bp fragment encoding the mature clKAS III, respectively) and 2.5 U proof-reading DNA polymerase. The DNA amplification was performed with an initial denaturation for 2 minutes at 94° C., followed by 30 cycles of denaturation for 0.5 minutes, annealing for 1 minute at 52° C. and elongation for 2.5 minutes at 72° C., followed by a final elongation step of 10 minutes at 72° C.

The resulting 204 bp and 1017 bp fragments, which overlap for a length of 6 base pairs, were used as template in a second PCR. The reaction mix contained 200 μM dNTPs, 50 μMol of each of the flanking primers clprae-5 and clcterm-3, 2 ng of each of the DNA fragments amplified in the first PCR reactions, and 2.5 U proof-reading Pfu DNA polymerase within a final volume of 50 μl. The employed temperature program for the DNA amplification in full length was as follows: initial denaturation for 3 minutes at 94° C., followed by 30 cycles of denaturation for 0.5 minutes, annealing for 1 minute at 55° C. and elongation for 3 minutes at 72° C., followed by a final elongation step for 10 minutes at 72° C.

After ligation of the resulting 1209 bp DNA into a sequencing vector the nucleotide sequence was verified by automated DNA sequencing.

b) Site-Specific Mutagenesis of the clKAS III

Asparagine³⁵⁸ of clKAS III (the position 358 is based on the protein sequence including the pre-peptide and corresponds to asparagine²⁹¹ of the mature cwKAS III) was substituted by aspartate by means of PCR-based site-specific mutagenesis using the “Quick-Change” kit of Stratagene (Heidelberg, Germany) according to the manufacturer's protocol. The desired mutation was introduced using a sense mutant primer (Clmut-sense, see Table 5) and an anti-sense mutant primer (Clmut-antisense, see Table 5), and the entire plasmid comprising the mutagenised clKAS III cDNA was amplified by PCR.

The reaction conditions for the amplification were as follows: 250 μM dNTPs, 100 ng wild-type clKAS III plasmid, 25 pMol of each of the sense and anti-sense mutant primer and 2.5 U proof-reading Pfu DNA polymerase in a final volume of 50 μl. The following optimised temperature program was employed: initial denaturation for 2 minutes at 95° C., 30 cycles of denaturation for 0.75 minutes, annealing for 1 minutes at 67° C., elongation for 9 minutes at 72° C.

The methylated basic template plasmid was digested with methyl DNA specific restriction endonuclease DpnI for one hour at 37° C. and the nicked PCR-amplified mutant plasmid, which is not methylated and therefore resistant to DpnI digestion, was used to transform competent E. Coli cells.

Finally, the sequence of the mutant clKAS III cDNA was verified by automated DNA sequencing (see SEQ ID NO: 5).The deduced amino acid sequence of clKAS III N358D is shown in SEQ ID NO: 6.

TABLE 5
Primer sequences as used for PCR in Examples 5
and 6

| Primer | Sequence ^{a, b} |
|---|---|
| NotI dT₁₈ | 5′-AACTGGAAGAATTCGCGGCCGCAGGAAT-3′ |
| 5a | 5′-ATGGCNAAYGCNTYNGGSTT-3′ |
| 3a | 5′-ATYCTCTGRTTNGCYTGRTG-3′ |
| 5b | 5′-GAYGTNGAYATGGTNYTNATG-3′ |
| 3b | 5′-AYAATNGCNCCCCANGT-3′ |
| NotI dT₁₈ | 5′-TTCCTGCGGCCGCGAATTCTTCCAGTT-3′ |
| adapter | |
| Cl-3′ RACE | 5′-CATAGCGATGGAGATGGGCAA-3′ |
| Bn-3′ RACE | 5′-CATTCAGATGGCGATGGTCAG-3′ |
| Cl 5′-NdeI | 5′-CATATGAGAGGATGCAAATTG-3′ |
| Cl 3′-XhoI | 5′-CTCGAGTCATCCCCATCTGAC-3′ |
| Bn 5′-NdeI | 5′-CATATGCGCGGTTGCAAGCTA-3′ |
| Bn 3′-XhoI | 5′-CTCGAGTCAACCCCACTTGAC-3′ |
| Clprae-5 | 5′-ATGGCGAATGCTTTGGGGTT-3′ |
| Clcterm-3 | 5′-TCATCCCCATCTGACAATGG-3′ |
| Cloverl-5 | 5′-GTGAGTAGAGGATGCAAATTG-3′ |
| Cloverl-3 | 5′-TCCTCTACTCACAAACCTCGG-3′ |
| Clmut-sense | 5′-CTTGGCGAATTATGGGGACACAAGCGCTGCATC |
| Clmut- | 5′-GATGCAGCGCTTGTGTCCCCATAATTCGCCAAG- |
| antisense | |

^{a}mutagenised codons are bold,
^{b}recognition sites for NdeI and XhoI restriction endonucleases are underlined.
Abbreviations:
cl = Cuphea lanceolata
cw = Cuphea wrightii
bn = Brassica napus

### DESCRIPTION OF THE FIGURES

FIG. 1: Sequence alignment of the KAS III primary structures including those of the pre-peptides

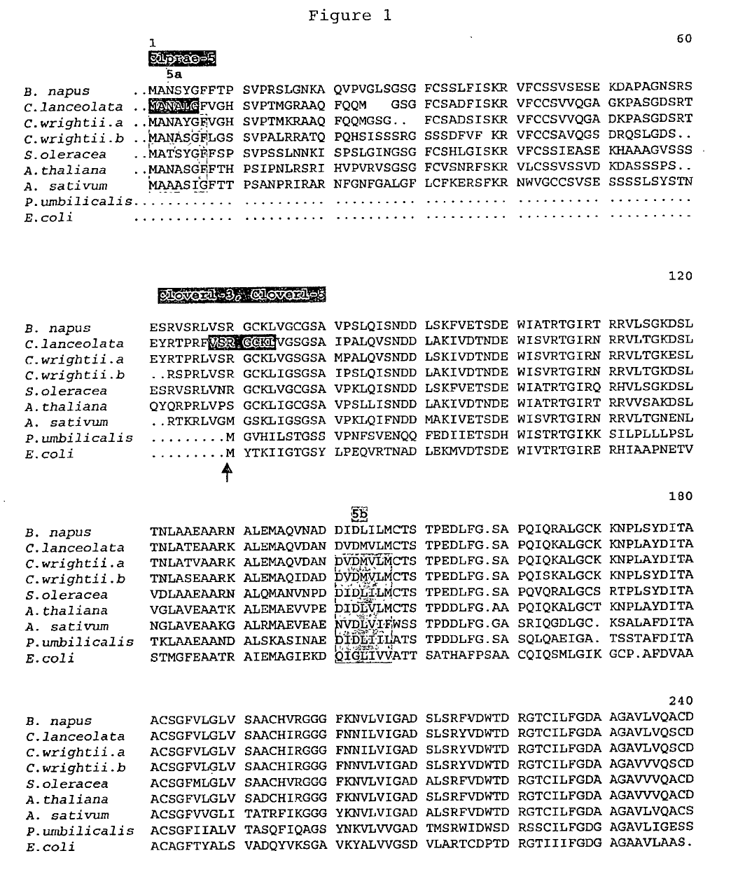

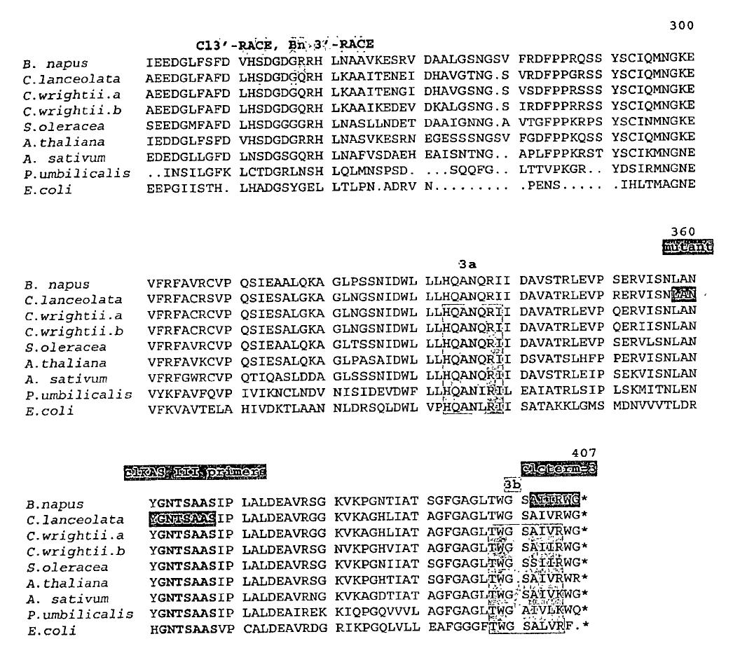

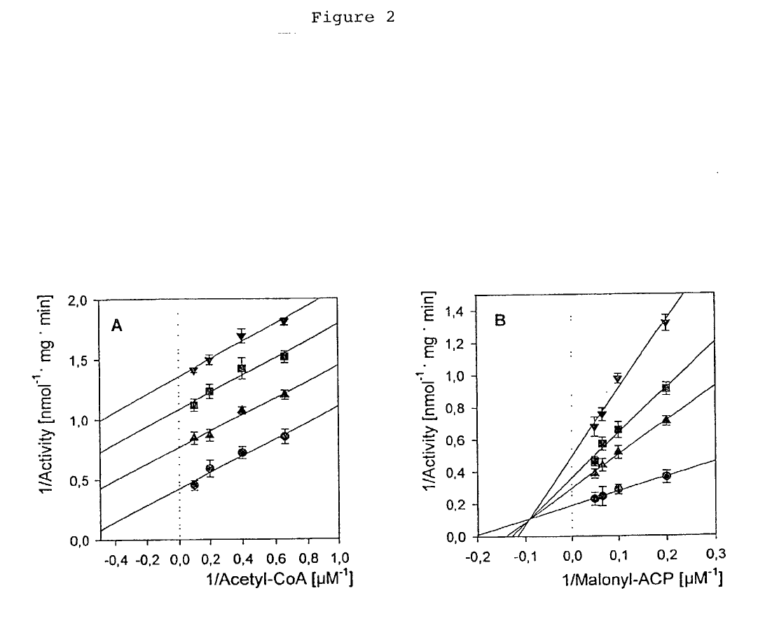

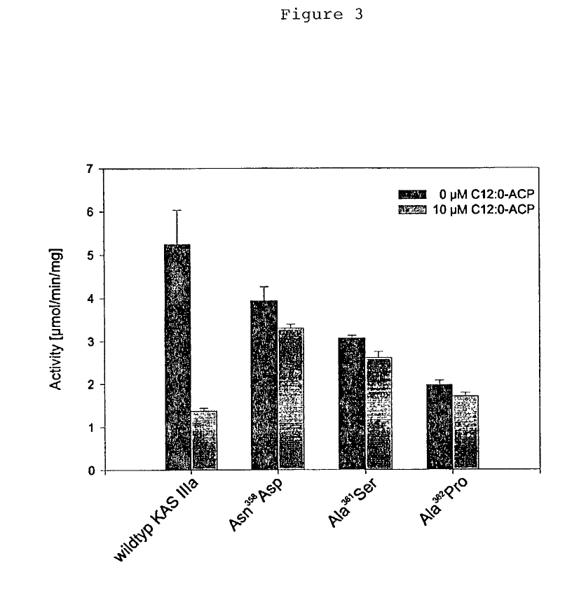

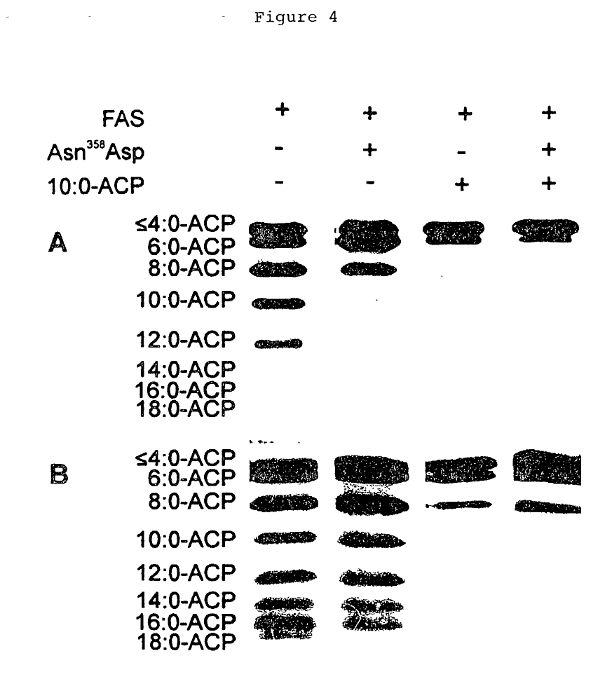

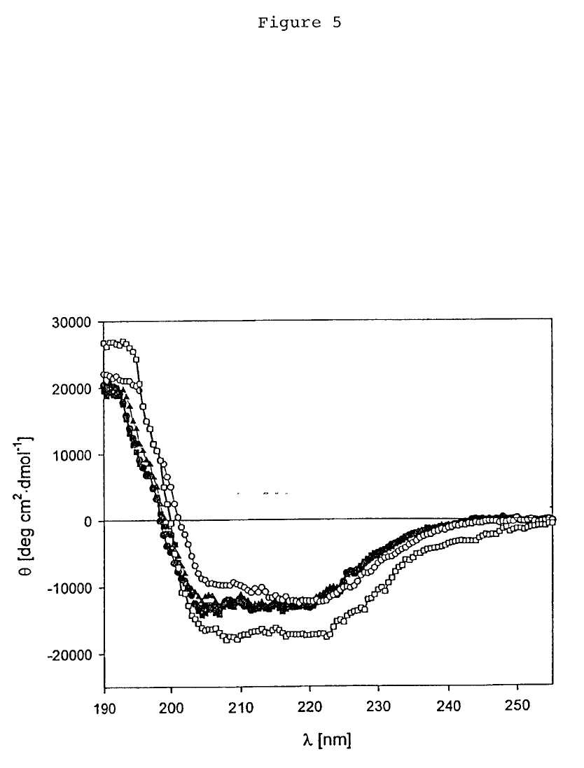

Regulatory sites are in bold. Sequence regions for the construction of PCR primers for the cloning of KAS III from C. lanceolata and B. napus are set in a light grey background. Target regions for the genetic engineering of clKAS III are in a dark grey background. The arrow shows the start of the mature protein and asterisks indicate the stop codon.

The references and Accession Nos. for the respective sequences are as follows, as far as they already belong to the prior art:

Cuphea wrightii; GenBank Accession No. U15935 (cwKAS IIIa); U15934 (cwKAS IIIb)

Slabaugh, M. B., Tai, H., Jaworski, J. G. and Knapp, S. J. (1995) cDNA clones encoding β-ketoacyl-acyl carrier protein synthase III from Cuphea wrightii. Plant Physiol.: 108, 343-444.

Spinacia oleracea; EMBL Accession No. Z22771

Tai, H. and Jaworski, J. G (1993) 3-Ketoacyl-Acyl Carrier Protein Synthase III from Spinach (Spinacia oleracea) is not similar to other Condensing Enzymes of Fatty Acid Synthase. Plant Physiol.: 103, 1361-1367.

Arabidopsis; GenBank Accession No. L31891

Tai, H., Post-Beittenmiller, D. and Jaworski, J. G. (1994) Cloning of a cDNA encoding 3-ketoacyl-acyl carrier protein synthase III from Arabidopsis. Plant Physiol.: 108, 343-444.

Allium sativum; GenBank Accession No. U306000

Chen, J. and Post-Beittenmiller, D. (1996) Molecular cloning of a cDNA encoding 3-ketoacyl-acyl carrier protein synthase III from leek. Gene: 182, 45-52.

Porphyra umbilicalis; GenBank Accession No. 438804

Reith, M. (1993) A β-ketoacyl acyl carrier protein synthase III gene (fabh) is encoded on the chloroplast of the red alga Porphyra umbilicalis. Plant Mol. Biol.: 21, 185-189.

Escherichia coli; GenBank Accession No. M77744

Tsay, J. T., Oh, W., Larson, T. J., Jackowski, S. and Rock, C.O. (1992) Isolation and characterization of the β-ketoacyl acyl carrier protein synthase III gene (fabH) from Escherichia coli K-12. J Biol. Chem.: 267, 6807-6814.

FIG. 2: Kinetic of the inhibition of wild-type KAS III by dodecanoyl ACP. Double reciprocal plots of the concentration of acetyl CoA (A) and malonyl ACP (B) against the activity of KAS III in the absence (●) and presence of 1 μM (▴), 2.5 μM (▪) and 5 μM (▾) dodecanoyl ACP. The respective substrate partner was maintained at a constant level at 10 μM [1-¹⁴C] acetyl CoA and 20 μM malonyl ACP. The enzyme activity was determined by monitoring the incorporation of [1-¹⁴C] acetate from [1-¹⁴C] acetyl CoA into β-ketobutyryl ACP (n=8).

FIG. 3: Inhibition of KAS III mutants by dodecanoyl ACP. The enzyme activity was determined by monitoring the incorporation of [1-¹⁴C] acetate from [1-¹⁴C] acetyl CoA into β-ketobutyryl ACP in the presence of 10 μM non-radioactive dodecanoyl ACP (n=4).

FIG. 4: Supplementation assays of FAS extracts from C. lanceolata seeds (A) and rapeseeds (B). The FAS reactions of the FAS preparations were supplemented with the KAS IIIa mutant Asn³⁵⁸Asp and 10 μM decanoyl ACP as shown. The control reactions were performed without addition of exogenous KAS IIIs. The reaction products were determined by the incorporation of [1-¹⁴C] acetate from [1-¹⁴C] acetyl CoA into acyl ACPs. Samples were collected after 20 minutes and were analysed by separating the acyl ACPs in a 5.0 M urea PAGE, followed by electro-blotting on Immobilon P and visualising by auto-radiography. The acyl residues are defined by the number of carbon atoms: number of double bonds (n=3).

FIG. 5: CD-spectra of the wild-type KAS IIIa (●), Asn³⁵⁸Asp (▪), Ala³⁶¹Ser (▴), Ala³⁶²Pro (O) and the deletion mutant (▾). Ellipticity (θ) is plotted against the wave length (λ).

What is claimed is:

1. A DNA sequence which codes for a protein having the enzymatic activity of a β-ketoacyl ACP synthase III (KAS III), wherein the protein is not controllable, especially not inhibited, by acyl ACPs.

2. The DNA sequence according to claim 1, which is altered compared to the wild-type sequence of KAS III by at least one mutation within the region encoding the amino acid sequence motif GNTSAAS.

3. The DNA sequence according to claim 1 or claim 2, wherein the mutation within the amino acid motif GNTSAAS of KAS III leads to substitution of the amino acid N by D and/or of the amino acid A (first alanine of the motif) by S.

4. The DNA sequence according to any of claims 1 to 3, which codes for a protein having the enzymatic activity of a β-ketoacyl ACP synthase III (KAS III) from Brassica napus, Cuphea lanceolata or Cuphea wrightii.

5. The DNA sequence according to any of claims 1 to 4, selected from the group consisting of
a) DNA sequences comprising a nucleotide sequence, which encode the amino acid sequence identified in SEQ ID NO: 6, or fragments thereof,
b) DNA sequences comprising the nucleotide sequence identified in SEQ ID NO: 5, or parts thereof,
c) DNA sequences comprising a nucleotide sequence, which hybridises to a complementary strand of the nucleotide sequence of a) or b), or parts of said nucleotide sequence,
d) DNA sequences comprising a nucleotide sequence, which is degenerate to a nucleotide sequence of c), or parts of said nucleotide sequence,
e) DNA sequences, which represent a derivative, analogue or fragment of a nucleotide sequence of a), b), c) or d).

6. A recombinant nucleic acid molecule, comprising
a) a promoter region,
b) a DNA sequence according to any of claims 1 to 5, which is operatively linked thereto, and
c) optionally, regulatory sequences, which are operatively linked thereto and may act as transcription, termination and/or polyadenylation signals in plant cells.

7. The recombinant nucleic acid molecule according to claim 6, wherein the nucleic acid sequence is in combination with a promoter that is active in plants.

8. The recombinant nucleic acid molecule according to claim 6 or claim 7, wherein the nucleic acid sequence is in combination with a promoter that is active in triacylglycerols synthesising or storing tissue.

9. The recombinant nucleic acid molecule according to any of claims 6 to 8, wherein the nucleic acid sequence further is in combination with enhancer sequences, sequences encoding leader peptides and/or other regulatory sequences.

10. A vector comprising a DNA sequence according to any of claims 1 to 5 or a recombinant nucleic acid molecule according to any of claims 6 to 9.

11. A recombinant protein having the enzymatic activity of a β-ketoacyl ACP synthase III (KAS III), wherein the protein is not controllable, especially not inhibited, by acyl ACPs.

12. The recombinant protein according to claim 11, originating from Cuphea lanceolata.

13. The recombinant protein according to claim 11, originating from Cuphea wrightii or Brassica napus.

14. The recombinant protein according to claim 12, having the amino acid sequence identified in SEQ ID NO: 6.

15. Transgenic plants and micro-organisms containing a DNA sequence according to any of claims 1 to 5 or a recombinant nucleic acid molecule according to any of claims 6 to 9.

16. The plants and micro-organisms according to claim 15, having an altered fatty acid content and/or an altered fatty acid composition compared to wild-type plants and wild-type micro-organisms, respectively.

17. The plants and micro-organisms according to claim 15 or claim 16, having an increased content of middle chain fatty acids compared to wild-type plants and wild-type micro-organisms, respectively.

18. The plants and micro-organisms according to any of claims 15 to 17, having an increased content of short chain fatty acids compared to wild-type plants and wild-type micro-organisms, respectively.

19. The plants according to any of claims 15 to 18, which are oil seed plants, especially rapeseed, sunflower, soybean, peanut, coconut, cotton, flax.

20. The micro-organisms according to any of claims 15 to 18, which are bacteria or algae.

21. A method for increasing the content of short chain and/or middle chain fatty acids in plants, especially in triacylglycerols synthesising and/or storing tissues, comprising the steps:
a) producing a nucleic acid sequence, which codes for a protein having the enzymatic activity of a KAS II, wherein the protein is not controllable, especially not inhibited, by acyl ACPs, and which comprises at least the following components which are successively arranged in 5′-3′ orientation,
a promoter, which is active in plants,
at least one nucleic acid sequence according to any of claims 1 to 5, and
optionally, a termination signal for transcription termination and addition of a poly(A) tail to the corresponding transcript, as well as, optionally, DNA sequences, derived therefrom,
b) transferring the nucleic acid sequence from a) to plant cells, and
c) optionally, regenerating completely transformed plants, and, if desired, propagating the plants.

22. A method for increasing the content of short and/or middle chain fatty acids in micro-organisms comprising the steps:
a) producing a nucleic acid sequence, which codes for a protein having the enzymatic activity of a KAS III, wherein the protein is not controllable, especially not inhibited, by acyl ACPs, and which comprises at least the following components which are successively arranged in 5′-3′ orientation,
a promoter, which is active in the respective micro-organism,
at least one nucleic acid sequence according to any of claims 1 to 5, and
optionally, a termination signal for transcription termination, and addition of a poly(A) tail to the corresponding transcript, as well as, optionally, DNA sequences, derived therefrom, and
b) transferring the nucleic acid sequence from a) to the respective micro-organism.

23. The method according to claim 21 or claim 22, in which between step a) and b) the acyl ACP binding site of the β-ketoacyl ACP synthase m is knocked out by in vivo mutation.

24. Use of plants or micro-organisms produced according to claim 21, for obtaining fatty acids and oils having an increased content of short and/or middle chain fatty acids.
